(19) United States
(12) Patent Application Publication
(10) Pub. No.: US 2015/0259285 A1
(43) Pub. Date: Sep. 17, 2015

(54) PROCESS TO PRODUCE ATORVASTATIN INTERMEDIATES

(71) Applicant: DSM SINOCHEM PHARMACEUTICALS NETHERLANDS B.V., Delft (NL)

(72) Inventors: Ben De Lange, Echt (NL); Dennis Heemskerk, Echt (NL); Henricus Leonardus Marie Elsenberg, Echt (NL)

(21) Appl. No.: 14/428,153

(22) PCT Filed: Sep. 12, 2013
(86) PCT No.: PCT/EP2013/068886

§ 371 Date: Mar. 13, 2015

(30) Foreign Application Priority Data
Sep. 17, 2012 (EP) 12184689.3

(51) Int. Cl. C07D 207/34

(52) U.S. Cl. CPC C07D 207/34

(57) ABSTRACT

The invention provides a process for the production of 1 a compound of formula (I), said process comprising reacting a compound of formula (II), with a compound of formula (III), wherein R1 and R2 may be the same or different and are selected from H; a C1-C6 alkyl which may be straight or branched, substituted or unsubstituted; or R1 and R2 together represent an alkylidene group of the formula CRaRb wherein Ra and Rb may be the same or different and are selected from an alkyl group having between 1 and 6 atoms, and wherein R3 represents a C1-C6 alkyl group, wherein the reaction is carried out in the presence of a base. Using a base may advantageously allow a lower temperature and/or may result in a higher yield. Also, a phase separation step may be omitted.

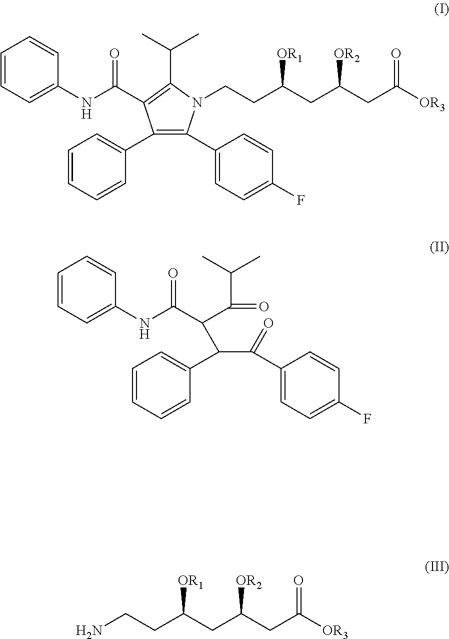

### FIELD OF THE INVENTION

c.

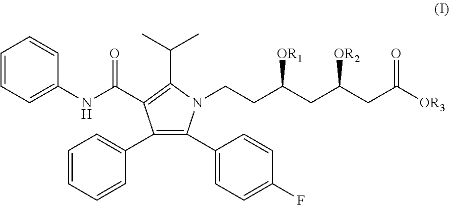

### BACKGROUND OF THE INVENTION

Atorvastatin ([R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium salt, (formula IV) is a pharmaceutical ingredient useful as an inhibitor of the enzyme 3-hydroxy-3-methylglutaryl-coenzyme A reductase (HMG-CoA reductase) and thus useful as a hypolipidemic and hypocholesterolemic agent.

Atorvastatin may be produced via the intermediate of formula I, which may be produced in a Paal-Knorr type condensation reaction involving reacting a compound of formula II

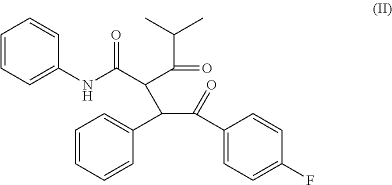 [SMILES: CC(C)C(=O)C(C(=O)Nc1ccccc1)C(C(=O)c1ccc(F)cc1)c1ccccc1]

and a compound of formula III

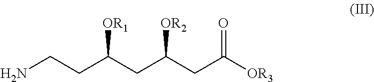

wherein R1 and R2 may be the same or different and are selected from H; a C1-C6 alkyl which may be straight or branched, substituted or unsubstituted; or R1 and R2 together represent an alkylidene group of the formula CRaRb wherein Ra and Rb may be the same or different and are selected from an alkyl group having between 1 and 6 atoms, and wherein R3 represents a C1-C6 alkyl group.

Such reaction is described in WO2006097909. A disadvantage of said reaction is that the yield is insufficient. Another disadvantage is that the reaction temperature is too high.

### DETAILED DESCRIPTION OF THE INVENTION

Therefore, the invention provides a process for the production of a compound of formula I,

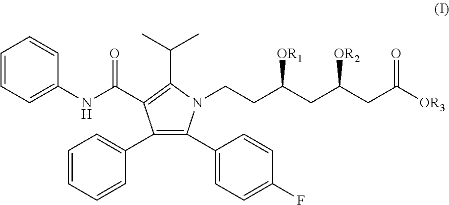

said process comprising reacting a compound of formula II

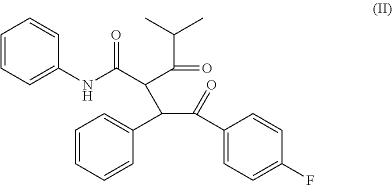 [SMILES: CC(C)C(=O)C(C(=O)Nc1ccccc1)C(C(=O)c1ccc(F)cc1)c1ccccc1]

with a compound of formula III

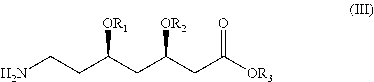

wherein R1 and R2 may be the same or different and are selected from H; a C1-C6 alkyl which may be straight or branched, substituted or unsubstituted; or R1 and R2 together represent an alkylidene group of the formula CRaRb wherein Ra and Rb may be the same or different and are selected from an alkyl group having between 1 and 6 atoms, preferably Ra and Rb are methyl or form a cyclohexyl or cyclopentyl, group, most preferably Ra and Rb are methyl, and wherein R3 represents a C1-C6 alkyl group, preferably R3 is isopropyl, 2-butyl, cyclohexyl or tert-butyl, most preferably isopropyl, wherein said reaction is carried out in the presence of a base.

The reaction of the invention is known to the skilled person as a Paal-Knorr condensation type reaction.

The inventors have surprisingly found that using a base may allow for a lower reaction temperature and/or may result in a higher yield. Also, a phase separation step may be omitted.

The base may be added to the process in any order. For example it may be added together with the other reactants.

The base may comprise a secondary amine. The skilled person would not be motivated a secondary amine to include in the process because secondary amines can react with ketone functional groups which are present in the compound of formula II and lead to unwanted side-products. Bases which can be used include cyclic or non-cyclic secondary aliphatic, aromatic or heteroaromatic amines such as diethylamine, piperidine, morpholine and di-isopropyl amine. A preferred base comprises di-isopropyl amine.

The compound of formula II may comprise 2-[2-(4-fluorophenyl)-2-oxo-1-phenylethyl]-4-methyl-3-oxopentanoic acid phenylamide.

The compound of formula III may comprise an amine (i.e. as a free amine) or an organic or inorganic acid thereof, or mixtures thereof. Said compound may comprise an organic acid salt or inorganic acid salt of (4R, 6R)-1,3-dioxane-4-acetic acid, 6-(2-aminomethyl)-2,2-dimethyl-,1-methylethylester. Preferably the compound of formula III comprises an organic acid salt, more preferably pivalic acid salt.

The reaction temperature and reaction time are such that the compound of formula I is produced. Suitable reaction temperatures may be between 50 and 100° C., preferably they range between 55 and 75° C., more preferably between 60 and 70° C. The temperature is preferably 95° C. or less, more preferably 90° C. or less, 85° C. or less, even more preferably 80° C. or less, most preferably it is 75° C. or less. The reaction time is less critical and may range e.g. between 10 and 100 hrs. Generally, at higher temperatures the reaction will proceed faster, whilst lower reaction times require longer reaction times. The skilled person can easily monitor the formation of the compound of formula I during the reaction, for example by HPLC, in order to determine when the reaction is completed.

The reaction may be carried out in the presence of cyclohexane and/or N-methyl-pyrrolidone. Adding cyclohexane and/or N-methyl-pyrrolidone may results in a higher yield and/or may shorten the reaction time and/or facilitate the isolation,

The reaction may be carried out at a reduced pressure, i.e. below atmospheric pressure. Carrying out the reaction at reduced pressure may result in an even further increase of the yield and or reduction in reaction time. Preferably the reaction pressure is between 500-700 Torr, even more preferably between 600-650 Torr, preferably the reaction is carried out under vacuum. Torr is the generally accepted unit of pressure used in industrial processes and is well known to the person skilled in the art. The conversion is such that 100,000 Pa corresponds to 750.06 torr.

The process of the invention is particularly suitable for the production of the atorvastatin intermediate ((4R,6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid 1-methylethyl ester if the compound of formula II is an ester of 2-[2-(4-fluorophenyl)-2-oxo-1-phenylethyl]-4-methyl-3-oxopentanoic acid phenylamide and if the compound of formula III is an organic acid salt or inorganic acid salt of (4R, 6R)-1,3-dioxane-4-acetic acid, 6-(2-aminomethyl)-2,2-dimethyl-,1-methylethylester. The atorvastatin intermediate can be used to produce atorvastatin hemi calcium salt. The intermediate may be isolated before it is converted to atorvastatin. The skilled person knows how to isolate said intermediate. For example, the isolation may include phase separation, for example using cyclohexane phase, precipitation, washing, e.g. using water/2-propanol, filtration etc.

The invention further provides a process to produce atorvastatin hemi calcium salt of formula IV,

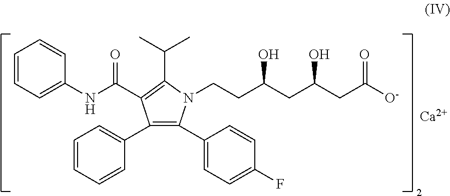

said process comprising the steps of:

(a) treating a solution of the atorvastatin intermediate produced in the process of the invention in a first solvent with an acid;

(b) treating the mixture obtained in step (a) with an alkali metal hydroxide;

(c) treating the mixture obtained in step (b) with a calcium salt or with calcium hydroxide.

The following examples are for illustrative purposes only and are not to be construed as limiting the invention.

### EXAMPLES

### Comparative Example A

### Preparation of 2-((4R, 6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid 1-methylethyl ester from the pivalic acid salt of (4R, 6R)-1,3-dioxane-4-acetic acid, 6-(2-aminomethyl)-2,2-dimethyl-,1-methylethylester and 2-[2-(4-fluorophenyl)-2-oxo-1-phenylethyl]-4-methyl-3-oxopentanoic acid phenylamide (DKT)

A reactor was charged with cyclohexane (800 mL), DKT (121 g, 0.29 mol), the pivalic acid salt of (4R, 6R)-1,3-dioxane-4-acetic acid, 6-(2-aminomethyl)-2,2-dimethyl-1-methylethylester (100 g, 0.28 mol) and N-methyl-pyrrolidone (50 mL). The reaction mixture was heated to reflux under azeotropic water removal for 48 h at 80-82° C. After cooling to 50-55° C., 10% aqueous NaHCO₃ (400 mL) was added and the reaction mixture stirred for 15 minutes. The phases were separated and the cyclohexane phase washed again with 10% aqueous NaHCO₃ (2×400 mL). The cyclohexane phase was then washed with water (2×200 mL). After phase separation, the cyclohexane solution was concentrated under vacuum to give an oily residue and stripped with 2×50 mL of 2-propanol. To the residue was added 2-propanol (450 mL) and heated to 75-80° C. to give a clear solution. Upon cooling to 50-55° C., the product precipitated and the slurry was further cooled to 40° C. Water (150 mL) was added and the slurry cooled to 20° C. After stirring for 10 h, the product was isolated by filtration and washed with 2-propanolwater (100 mL, 3/1 v/v). The wet-cake was added to 2-propanol (450 mL), heated to 80° C. until a clear solution was obtained. The solution was cooled to 30° C. Water (150 mL) was added and the slurry cooled to 20° C. After stirring for 16 h, the product was isolated by filtration and washed with 2-propanolwater (100 mL, 3/1 v/v). The wet-cake was added to 2-propanol (400 mL), heated to 80° C. until a clear solution was obtained. The solution was cooled to 20° C. After stirring for 16 h, the product was isolated by filtration and washed with 2-propanol (50 mL). The product was dried under vacuum (105.5 g, 0.165 mol, 58.9% yield based on the pivalic acid amine salt) and analysed by HPLC.

### Example 1

### Preparation of 2-((4R, 6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid 1-methylethyl ester from the pivalic acid salt of (4R, 6R)-1,3-dioxane-4-acetic acid, 6-(2-aminomethyl)-2,2-dimethyl-,1-methylethylester and 2-[2-(4-fluorophenyl)-2-oxo-1-phenylethyl]-4-methyl-3-oxopentanoic acid phenylamide (DKT)

A reactor was charged with cyclohexane (400 mL), DKT (112 g, 0.27 mol), the pivalic acid salt of (4R, 6R)-1,3-dioxane-4-acetic acid, 6-(2-aminomethyl)-2,2-dimethyl-1-methylethylester (100 g, 0.28 mol), N-methyl-pyrrolidone (50 mL) and di-isopropyl amine as a base (38 mL, 27.4 g, 0.27 mol). The reaction mixture was heated to reflux under azeotropic water removal for 48 h at 80-82° C. After cooling, the cyclohexane solution was concentrated under vacuum to give an oily residue and stripped with 2×50 mL of 2-propanol. To the residue was added 2-propanol (450 mL) and heated to 75° C. to give a clear solution. Upon cooling to 45-50° C., the product precipitated. Water (150 mL) was added and the slurry cooled to 20° C. After stirring for 16 h, the product was isolated by filtration and washed with 2-propanolwater (100 mL, 3/1 v/v). The wet-cake was added to 2-propanol (450 mL), heated to 80° C. until a clear solution was obtained. The solution was cooled to 30° C. Water (150 mL) was added in 1 h and the slurry cooled to 20° C. After stirring for 16 h, the product was isolated by filtration and washed with 2-propanolwater (100 mL, 3/1 v/v). The wet-cake was added to 2-propanol (400 mL), heated to 80° C. until a clear solution was obtained. The solution was cooled to 20° C. After stirring for 16 h, the product was isolated by filtration and washed with 2-propanol (2×25 mL). The product was dried under vacuum (111.0 g, 0.173 mol, 61.8% yield based on the pivalic acid amine salt) and analysed by HPLC.

### Example 2

### Preparation of 2-((4R, 6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid 1-methylethyl ester from the pivalic acid salt of (4R, 6R)-1,3-dioxane-4-acetic acid, 6-(2-aminomethyl)-2,2-dimethyl-,1-methylethylester and 2-[2-(4-fluorophenyl)-2-oxo-1-phenylethyl]-4-methyl-3-oxopentanoic acid phenylamide (DKT)

A reactor was charged with cyclohexane (400 mL), DKT (112 g, 0.27 mol), the pivalic acid salt of (4R, 6R)-1,3-dioxane-4-acetic acid, 6-(2-aminomethyl)-2,2-dimethyl-1-methylethylester (100 g, 0.28 mol) and N-methyl-pyrrolidone (40 mL). The reaction mixture was heated to reflux under vacuum (600 mbar) and under azeotropic water removal for 60 h at 68-70° C. After cooling, the cyclohexane solution was concentrated under vacuum to give an oily residue and stripped with 2×50 mL of 2-propanol. To the residue was added 2-propanol (450 mL) and heated to 75° C. to give a clear solution. After cooling to 70° C., water (150 mL) was added, whereupon the product crystallized. The reaction mixture was 20°. After stirring for 16 h, the product was isolated by filtration and washed with 2-propanolwater (100 mL, 3/1 v/v). The wet-cake was added to 2-propanol (450 mL), heated to 80° C. until a clear solution was obtained. The solution was cooled to 40° C. Water (150 mL) was added and the slurry cooled to 20° C. After stirring for 16 h, the product was isolated by filtration and washed with 2-propanolwater (100 mL, 3/1 v/v). The wet-cake was added to 2-propanol (400 mL), heated to 80° C. until a clear solution was obtained. The solution was cooled to 20° C. After stirring for 16 h, the product was isolated by filtration and washed with 2-propanol (2×25 mL). The product was dried under vacuum (110.8 g, 0.173 mol, 61.7% yield based on the pivalic acid amine salt) and analysed by HPLC.

### Example 3

### Preparation of 2-((4R, 6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid 1-methylethyl ester from the pivalic acid salt of (4R, 6R)-1,3-dioxane-4-acetic acid, 6-(2-aminomethyl)-2,2-dimethyl-,1-methylethylester and 2-[2-(4-fluorophenyl)-2-oxo-1-phenylethyl]-4-methyl-3-oxopentanoic acid phenylamide (DKT)

A reactor was charged with cyclohexane (400 mL), DKT (112 g, 0.27 mol), the pivalic acid salt of (4R, 6R)-1,3-dioxane-4-acetic acid, 6-(2-aminomethyl)-2,2-dimethyl-1-methylethylester (100 g, 0.28 mol), N-methyl-pyrrolidone (25 mL) and di-isopropyl amine as a base (38 mL, 27.4 g, 0.27 mol). The reaction mixture was heated to reflux under vacuum (600 mbar) and under azeotropic water removal for 54 h at 68-70° C. After cooling, the cyclohexane solution was concentrated under vacuum and the residue stripped with 2×50 mL of 2-propanol. To the residue was added 2-propanol (450 mL) and heated to 75° C. to give a clear solution. After cooling to 55-60° C., precipitation started and the mixture cooled to 30° C., when water (150 mL) was added in 1 h. After cooling to 20° C., the reaction mixture was stirred for 16 h. The product was isolated by filtration and washed with 2-propanolwater (2×50 mL, 3/1 v/v). The wet-cake was added to 2-propanol (450 mL), heated to 80° C. until a clear solution was obtained. The solution was cooled to 30° C., water (150 mL) was added and the slurry cooled to 20° C. After stirring for 16 h, the product was isolated by filtration and washed with 2-propanol/water (100 mL, 3/1 v/v). The wet-cake was added to 2-propanol (400 mL), heated to 80° C. until a clear solution was obtained. The solution was cooled to 20° C. After stirring for 16 h, the product was isolated by filtration and washed with 2-propanol (2×25 mL). The product was dried under vacuum (116.9 g, 0.183 mol, 65.4% yield based on the pivalic acid salt) and analysed by HPLC.

1. Process for the production of a compound of formula I, 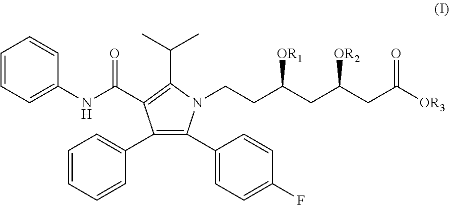
said process comprising reacting a compound of formula II 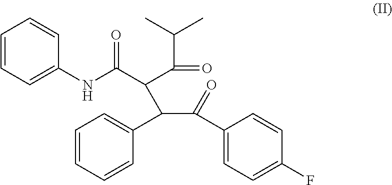 [SMILES: CC(C)C(=O)C(C(=O)Nc1ccccc1)C(C(=O)c1ccc(F)cc1)c1ccccc1]
with a compound of formula III 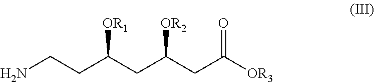
wherein R1 and R2 may be the same or different and are selected from H; a C1-C6 alkyl which may be straight or branched, substituted or unsubstituted; or R1 and R2 together represent an alkylidene group of the formula CRaRb wherein Ra and Rb may be the same or different and are selected from an alkyl group having between 1 and 6 atoms, and wherein R3 represents a C1-C6 alkyl group, wherein said reaction is carried out in the presence of a base.

2. Process according to claim 1 wherein the base comprises a secondary amine.

3. Process according to claim 1 wherein the base comprises di-isopropyl amine.

4. Process according to claim 1 wherein Ra and Rb are methyl or form a cyclohexyl or cyclopentyl group.

5. Process according to claim 1 wherein R3 is isopropyl, 2-butyl, cyclohexyl or tert-butyl.

6. Process according to claim 1 wherein the compound of formula II comprises 2-[2-(4-fluorophenyl)-2-oxo-1-phenylethyl]-4-methyl-3-oxopentanoic acid phenylamide.

7. Process according to claim 1 wherein the compound of formula III comprises an organic acid salt or inorganic acid salt of (4R, 6R)-1,3-dioxane-4-acetic acid, 6-(2-aminomethyl)-2,2-dimethyl-,1-methylethylester.

8. Process according to claim 1 wherein the compound of formula III comprises an organic acid salt, preferably pivalic acid salt.

9. Process according to claim 1 wherein the reaction is carried out in the presence of cyclohexane and/or N-methyl-pyrrolidone.

10. Process according to claim 1 wherein the reaction is carried out below atmosphere pressure.

11. Process according to produce the atorvastatin intermediate ((4R,6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid 1-methylethyl ester by the process according to claim 1 wherein the compound of formula II comprises an ester of 2-[2-(4-fluorophenyl)-2-oxo-1-phenylethyl]-4-methyl-3-oxopentanoic acid phenylamide and wherein the compound of formula III comprises an organic acid salt or inorganic acid salt of (4R, 6R)-1,3-dioxane-4-acetic acid, 6-(2-aminomethyl)-2,2-dimethyl-,1-methylethylester.

12. Process to produce atorvastatin hemi calcium salt of formula IV, 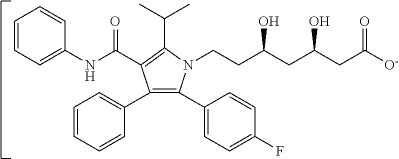 [SMILES: CC(C)c1c(C(=O)Nc2ccccc2)c(-c2ccccc2)c(-c2ccc(F)cc2)n1CC[C@@H](O)C[C@@H](O)CC(=O)[O-]]
said process comprising the steps of:
(a) treating a solution of the atorvastatin intermediate produced in the process of claim 11 in a first solvent with an acid;
(b) treating the mixture obtained in step (a) with an alkali metal hydroxide;
(c) treating the mixture obtained in step (b) with a calcium salt or with calcium hydroxide.
